(19) United States
(12) Patent Application Publication
(10) Pub. No.: US 2021/0353789 A1
(43) Pub. Date: Nov. 18, 2021

(54) SELF-DISINFECTING OBJECT

(71) Applicant: FRAUNHOFER-GESELLSCHAFT ZUR FÖRDERUNG DER ANGEWANDTEN FORSCHUNG E. V., München (DE)

(72) Inventor: Simon STIER, Würzburg (DE)

(73) Assignee: FRAUNHOFER-GESELLSCHAFT ZUR FÖRDERUNG DER ANGEWANDTEN FORSCHUNG E. V., München (DE)

(21) Appl. No.: 17/316,851

(22) Filed: May 11, 2021

(30) Foreign Application Priority Data
May 14, 2020 (DE) 10 2020 206 087.8

(51) Int. Cl. A61L 2/04; A61L 2/24; A61L 2/00

(52) U.S. Cl. CPC A61L 2/04; A61L 2/24; A61L 2202/24; A61L 2202/14; A61L 2202/21; A61L 2/0023

(57) ABSTRACT

The invention relates to a self-disinfecting object having a contact surface of a substrate provided for contact with a human body part, wherein the contact surface is at least regionally heatable.

### CROSS-REFERENCE TO RELATED APPLICATIONS

This patent application claims the benefit of German Patent Application No. 10 2020 206 087.8, filed on May 14, 2020, the disclosure of which is incorporated herein by reference in its entirety for all purposes.

### BACKGROUND OF THE INVENTION

The invention relates to a self-disinfecting object having a contact surface of a substrate provided for contact with a human body part, wherein the contact surface is at least regionally heatable.

A substantial main transmission route of pathogens is droplet infection via aerosols that are produced during coughing, for instance. Virus particles, however, also remain infectious for up to a plurality of days on metals, plastics, or glass in dependence on conditions and bacteria, in particular in their persistent form (spores), even considerably longer. A transmission can therefore also take place in this manner via a smear infection even if the affected persons never had a direct contact. In addition to measures on one's own responsibility such as washing hands and avoiding contact with mucous membranes, regular disinfection of often touched objects such as door knobs, hand rails, or control surfaces (switches, time-punch machines, etc.) is essential. Such measures are even more important in medical facilities in which multiresistant bacteria also represent a serious problem in addition to viral disease. Regular manual disinfection of surfaces is, however, associated with substantial personal effort; in addition, there is the risk of developing resistance with chemical active agents.

The provision of the surfaces with antibacterially and/or antivirally acting substances such as copper or silver is known from the prior art. The active spectrum of substances is restricted, however, and can also be limited in time by wear such as corrosion, abrasion, and active ingredient depletion.

A number of active systems have also been developed as alternatives to passive protective layers. Such active systems are known from the EP 3 118 395 A2, US 2014/0137369 A1, US 2010/0140499 A1, and DE 20 2014 006 908 U1. Applications can be seen herefrom in which disinfectants are released automatically or mechanically or surfaces of objects are irradiated with UV light. In this process, however, only a portion of the surface of the objects is treated or a complex mechanism, that is potentially susceptible to error, or substantial construction modifications to the object are required. These concepts can generally also not be transferred to other holding and control surfaces.

A further solution known from the prior art is a heat treatment for disinfection. WO 1995/019216, EP 1 438 263 A1, WO 2005/079948 and DE 4 206 901 A1 thus describe devices for the thermal disinfection of elements of fluid circuits.

However, separate heating elements are used or internal surfaces are heated in this process.

Heatable surfaces have previously above all been established for comfort functions in automobiles such as seat heating or steering wheel heating. They are, however, based on heating wires, coils, or coatings that heat the total volume body. The heating process is therefore very slow, energy intensive, and as a rule limited to low temperatures. In addition, such components cannot be retrofitted to existing surfaces. Examples for heated surface elements in the automotive sector are known from U.S. Pat. No. 7,291,815 B2 (composite ice protection for aircraft) and DE 10 2012 207 708 A1 (heating coating for vehicles). They are, however, intended for deicing or heating the interior space.

DE 10 2009 016 370 A1 describes a control element having a heated contact surface to disinfect it. However, the heating element is separate from the contact surface. US 2016/0317684 A1 describes a heatable self-disinfecting surface, wherein the energy supply is implemented via electromagnetic waves.

### BRIEF SUMMARY OF THE INVENTION

Starting from this, it is the object of the present invention to provide the surface of an object with a self-disinfecting coating to enable a disinfection of these objects that is simple to implement and that is automated.

This object is achieved by the self-disinfecting object having the features of the present invention and the advantageous further developments described herein.

### BRIEF DESCRIPTION OF THE SEVERAL VIEWS OF THE DRAWING(S)

The subject matter in accordance with the invention will be explained in more detail with reference to the following figures without intending to restrict it to the specific embodiments shown here.

FIG. 1 shows a disinfecting object in accordance with an embodiment of the invention;

FIG. 2A shows an overview of an irregularly shaped surface piece for a flat object in accordance with an embodiment of the invention;

FIG. 2B shows the sectional view (B-B) depicted in FIG. 2A;

FIG. 2C shows the sectional view (A-A) depicted in FIG. 2A;

FIG. 3A shows an overview of an irregularly shaped surface piece for a flat object with a segmented electrically conductive layer in accordance with another embodiment of the invention;

FIG. 3B shows the sectional view (C-C) depicted in FIG. 3A.

FIG. 3C shows the sectional view (B-B) depicted in FIG. 3A

FIG. 4A shows an overview of a cylindrical object in accordance with an embodiment of the invention;

FIG. 4B shows the side view with the depiction of the sections (A-A) and (B-B) of the object shown in FIG. 4A;

FIG. 4C shows the sectional view (B-B) depicted in FIG. 4B;

FIG. 4D shows the sectional view (A-A) depicted in FIG. 4B;

FIG. 5A shows an overview of a cylindrical object in accordance with another embodiment of the invention;

FIG. 5B shows the side view with the depiction of the sections (C-C) and (B-B) of the object shown in FIG. 5A;

FIG. 5C shows the sectional view (C-C) depicted in FIG. 5B;

FIG. 5D shows the sectional view (B-B) depicted in FIG. 5B;

FIG. 6 shows a further embodiment in accordance with the invention for a door handle.

The following reference numerals are used for all the Figures:
1 coated object
2 thermally insulating layer
3 electrically conductive layer
3a, 3b segments of the electrically conductive layer
4 electrically insulating layer
4a, 4b segments of the electrically insulating layer
5, 5a, 5b electrical contact(s)
6 control electronics with power source
7 pathogens on the contact surface
8 door fitting

### DETAILED DESCRIPTION OF THE INVENTION

In accordance with the invention, a self-disinfecting object is provided having a contact surface of the object provided for contact with a human body part, wherein the contact surface is at least regional. This is achieved in that at least one disinfection coating is arranged at least in regions of the contact surface. This disinfection coating includes at least one electrically conductive layer that is connected via at least two electrical contacts to at least one control electronic system for the resistive heating of the at least one electrically conductive layer.

Within the framework of the present invention, disinfecting or disinfection is generally to be understood as a reduction in the number of pathogens. It specifically comprises disinfection in its actual meaning, i.e. a reduction of pathogens by a factor of at least 10⁻⁵ and a sterilization, i.e. a reduction of pathogens by a factor of at least 10⁻⁷.

Any human body parts can generally be considered for contact with the object, with them preferably being body parts of different persons since disinfection is particularly important for such objects to avoid infections between a plurality of persons.

The following advantages are associated with the present invention:
the disinfection is effective against all pathogens (algae, bacteria, parasites, funguses, prions, protists, viruses, and viroids);
abrasion or wear of the disinfecting coating can be avoided;
the disinfection in accordance with the invention is maintenance-free;
the disinfection in accordance with the invention can take place fully automatically without human intervention; and
the object itself does not have to be modified construction-wise (apart from the disinfection coating used).

The time, the duration, and the temperature of the heating by the control electronics is preferably selected such that the proper use of the object is still provided and a sufficiently high number of pathogens on the surface are rendered harmless to reduce the transmission of such pathogens via the surface with respect to a non-heated variant of the surface. In longer usage breaks (at night for instance), a higher (approximately >120° C.) and longer heating can additionally be performed to e.g. also render heat-resistant spores harmless.

A preferred embodiment provides that the control electronics have at least one temperature sensor and/or at least one temperature regulation, with the temperature regulation preferably being adapted for a surface temperature of the object of at least 70° C., particularly preferably at least 120° C., and/or with operating conditions being reached in which the surface temperature amounts at least regionally to at least 70° C., particularly preferably at least 120° C.

Conventional voltage-based methods (regulation of the current via the voltage) or time-based methods (regulation of the power via duration of switching on and off, cf. pulse width modulation) can be used for the regulation of the heating power or of the achieved surface temperature by the control electronics.

It is preferred that the at least one disinfection coating has at least one electrically insulating layer between the surface remote from the object and, starting therefrom, the next (topmost) electrically conductive layer and/or has at least one thermally insulating layer between the surface facing the object and, starting from this, the next (bottommost) electrically conductive layer.

The at least one electrically conductive layer preferably consists of or comprises one of the following materials or combinations thereof:
metals, in particular selected from the group consisting of copper, aluminum, gold, silver, tin, nickel, iron, zinc, and alloys thereof;
carbon modifications, in particular selected from the group consisting of graphite, graphene, carbon fibers, carbon nanotubes, carbon black, and mixtures thereof;
electrically conductive ceramic materials, in particular selected from the group consisting of SiC, ZrC, TiC, WC, TiN, ZrN, TiB₂, ZrB₂, TiO, TiSi₂ or MoSi₂;
polymer materials comprising electrically conductive particles, wherein the electrically conductive particles are preferably selected from the group consisting of powders, nanowires, flakes, or dendrites of said metals, powders of the previously named ceramic materials and of the previously named carbon modifications.

The at least one electrically insulating layer preferably consists of or comprises one of the following materials or combinations thereof:
thermally conductive materials, in particular selected from the group consisting of boron nitride, aluminum oxide, and aluminum nitride;
polymer materials comprising particles of the previously named thermally conductive materials;
polymer materials comprising particles of the previously named electrically conductive materials, wherein their volume contents are below the percolation threshold.

The at least one thermally insulating layer preferably comprises or consists of one of the following materials or combinations thereof:
thermally insulating materials, in particular selected from the group consisting of silicate ceramics, silicone, polymethyl methacrylate, polystyrene, polyvinylchloride; glass wool or mineral wool;
polymer materials comprising particles of the previously named thermally insulating materials;
materials comprising pores, in particular foamed materials;
materials comprising cavities, in particular polymer materials, wherein the cavities are in particular hollow glass spheres or hollow microspheres having a polymer shell.

It is preferred in this process that said polymer materials are selected from the group consisting of silicone, inorganic-organic hybrid polymers such as organically highly modified polysiloxanes, fluorosilicone, polyurethane (PUR), ethylene propylene diene rubber, polychlorobutadiene (CR), acrylonitrilebutadiene (NBR), polyester resin, epoxy resin, acrylic resin, phenolic resin, amino resin, polyoxymethylenes (POMs), polypropylene (PP), acrylonitrile butadiene styrene copolymer (ABS), polycarbonate (PC), and polymethyl methacrylate (PMMA).

A preferred embodiment provides that the at least one electrically conductive layer has a uniform or locally variable layer thickness in the range of 0.1 to 1000 μm, preferably of 1 to 100 μm.

The at least one electrically insulating layer preferably has a uniform or locally variable layer thickness in the range from 0.1 to 500 μm, preferably of 1 to 10 μm.

It is preferred that the at least one thermally insulating layer has a uniform or locally varying layer thickness in the range from 0.05 to 10 mm, preferably of 0.1 to 1 mm.

It is preferred here that the heat capacity of the at least one electrically conductive layer is very small, e.g. due to a suitable material selection and small layer thicknesses, while the insulation by the at least one thermally insulating layer toward the object is kept very high so that little energy is required to reach and maintain the target temperature and no burns occur on an (accidental) contact with a human body part.

In an advantageous embodiment, the degree of emission of the topmost layer facing away from the object is still small to achieve small radiation losses and the thermal conductivity of an at least one electrically insulating layer is large to achieve a uniform heating.

In an advantageous embodiment, the layer thickness of the at least one electrically conductive layer is furthermore adapted to the geometry of the object or of the substrate such that a uniform heating takes place, e.g. in that the layer thickness is increased on a tapering of the at least one electrically conductive layer. Alternatively, this can also take place by a structuring of the at least one electrically conductive layer, e.g. in the form of a segmentation thereof into individual segments.

A further preferred embodiment provides that the at least one electrically conductive layer has at least two segments that are electrically insulated room one another and that are connected to control electronics via separate electrical contacts.

It is preferred that at least one of the electrically conductive layers is formed as a resistive, capacitive, or inductive sensor element for measuring distance, temperature, pressure, elongation, force, shear, bend, or wetting of the contact surface or serves as a proximity sensor for living beings. This can serve to recognize and localize a human presence and to respond accordingly (switch off heating, emit warning signal). In addition, biofilms that may be present or a manual cleaning with liquid cleaning agents that has taken place can, e.g., be determined by an evaluation of the impedance.

The at least one disinfection coating preferably has a transmission of at least 10%, preferably 20%. The objects can thus remain visually unchanged by such a transparent disinfection coating, in particular by transparent electrically conductive layers.

A further preferred embodiment provides that the at least one disinfection coating has at least one light element. Light elements such as an LED can thus, e.g., be integrated for visual feedback.

The disinfection coating is preferably elastic and particularly preferably has an elasticity of at least 5%, particularly preferably at least 20%.

The object in accordance with the invention is preferably deformable, e.g. a foam, bolster, or cushion. Deformable objects such as bolsters can thus also be provided by an elastic deformation coating, in particular an elastic electrically conductive layer.

It is preferred that the object is selected from the group consisting of
manually operable devices or mechanical holding and control devices, in particular door handles, hand rails, grab handles;
switching or control elements, in particular light switches, door openers, elevator control elements;
medical devices, in particular manually operable medical devices and tools, examination tables, operating tables;
sports apparatus, in particular fitness apparatus.

An embodiment is a door handle equipped in accordance with the invention. It is heated at suitable time intervals to disinfect the surface. The disinfection takes place, for example, periodically or when the control electronics determine a break in usage by a suitable sensor system (for instance by motion sensors or a proximity sensor system).

A further embodiment is an operating table equipped in accordance with the invention. It can be heated for disinfection, manually or automatically triggered between two operations. A temperature regulation of the surface for the comfort of the patient can furthermore also be achieved.

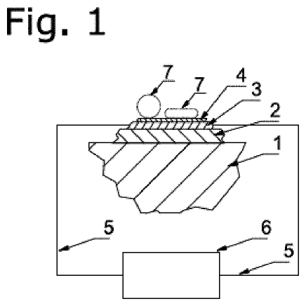

As shown in FIG. 1, an embodiment in accordance with the invention comprises a resistively heatable coating for the surface of an exposed object 1. In an advantageous embodiment, it comprises a thermally, and optionally also electrically, insulating layer 2, an electrically conductive layer 3, and an electrically insulating and optionally thermally conductive layer 4. Control electronics 6 conduct electric power as required via at least two electrical contacts 5 through the middle layer 3 to resistively heat it. Viruses, bacteria, bacterial spores, funguses, fungus spores, and other pathogens 7 are rendered harmless at a sufficiently high surface temperature (>70° C. as a rule).

Conventional voltage-based methods (regulation of the current via the voltage) or time-based methods (regulation of the power via duration of switching on and off, cf. pulse width modulation) can be used for the regulation of the heating power or of the achieved surface temperature by the control electronics.

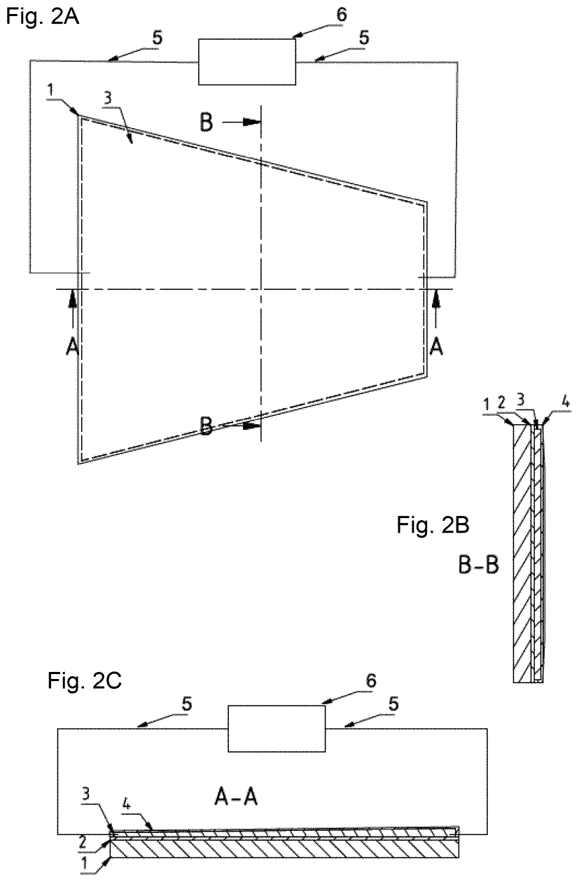

A further advantageous embodiment for a planar object is shown in FIGS. 2A-2C. FIG. 2A shows an overview of an exemplary, irregularly shaped surface piece. FIG. 2B shows the sectional view (B-B) depicted in FIG. 2A. FIG. 2C shows the sectional view (A-A) depicted in FIG. 2A. It can be seen here that the layer thickness of the conductive layer 3 has been provided with a layer thickness gradient to achieve a uniform heating, i.e. in this case that on a tapering of the conductive layer 3 its layer thickness is increased. Without this layer thickness gradient, i.e. with a homogeneous layer thickness, a greater heating would occur at the narrower end of the coated object 1 with a constant specific conductivity of the material used for the conductive layer 3 since the electrical voltage drop here and thus also the emitted power is higher here due to the greater electrical resistance.

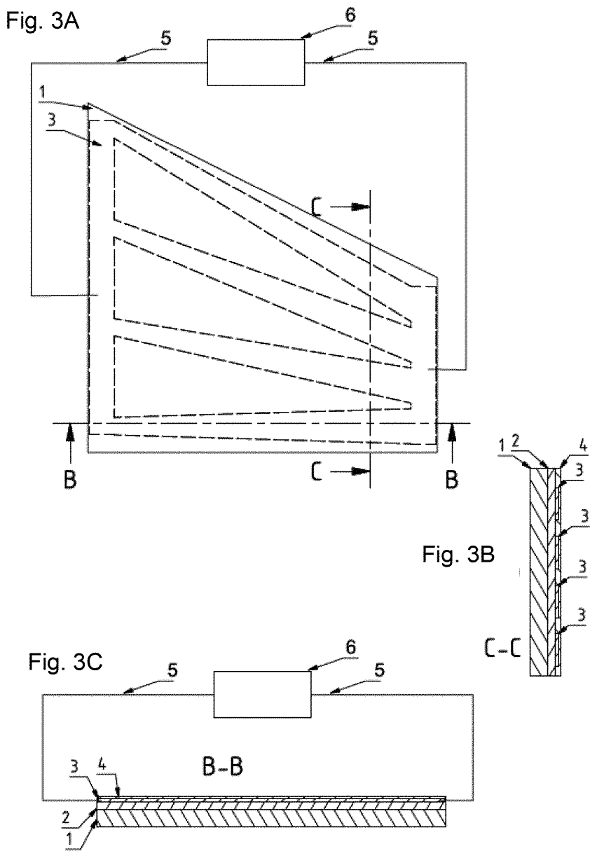

FIGS. 3A-3C show an alternative advantageous embodiment in which the uniform heating is achieved by a structuring of the conductive layer. FIG. 3A shows an overview of an exemplary irregularly shaped surface piece. It can be seen here that the electrically conductive layer 3 is divided into individual segments whose widths reduce as the width of the coated object 1 increases. Without this structuring, i.e. with a full surface coating with a homogeneous layer thickness, a greater heating would occur at the narrower end of the coated object 1 with a constant specific conductivity of the material used for the conductive layer 3 since the electrical voltage drop here and thus also the emitted power is higher due to the greater electrical resistance. The contact of the segments with the control electronics 6 could also take place via separate electrical contacts. This would have the advantage that the regulation of the heating power per segment can likewise take place separately. FIG. 3B shows the sectional view (C-C) depicted in FIG. 3A. FIG. 3C shows the sectional view (B-B) depicted in FIG. 3A.

An operating table can e.g. be provided with the advantageous embodiments shown in FIGS. 2A-2C and 3A-3C. It can be heated for disinfection, manually or automatically triggered between two operations. A temperature regulation of the surface for the comfort of the patient can also additionally be achieved. Other medical instruments and planar control elements in buildings such as light switches can furthermore also be equipped in this manner.

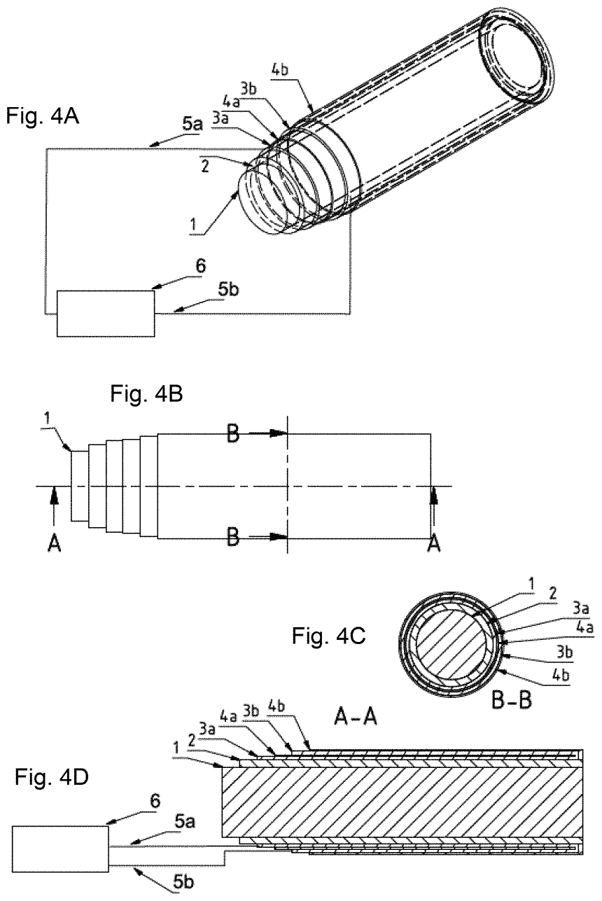

FIGS. 4A-4D show a further advantageous embodiment for a cylindrical object. FIG. 4A shows an overview of an exemplary cylindrical object, shown shortened here for reasons of the overview. The length is, however, generally freely selectable. Layers removed in steps at the end furthermore only serve the better understanding. The electrically conductive layer is divided into two layers 3a and 3b here. They are electrically insulated from one another by a further electrically insulating layer 4a. The connection to the control electronics takes place via the electrical contacts 5a and 5b respectively. As before, an electrically insulating layer is also located at the surface 4b. The side view with the definition of the sections (A-A) and (B-B) is shown in FIG. 4B. FIG. 4C shows the sectional view (B-B) depicted in FIG. 4B. FIG. 4D shows the sectional view (B-B) depicted in FIG. 4B. It can be seen here that the separated electrically conductive layers 3a and 3b are connected to one another at the end of the coated object 1 and the heating power circuit is thus closed. In comparison with an embodiment having only one electrically conductive layer, the electrical terminal 5 of the control electronics 6 can therefore advantageously only take place at one end of the object 1.

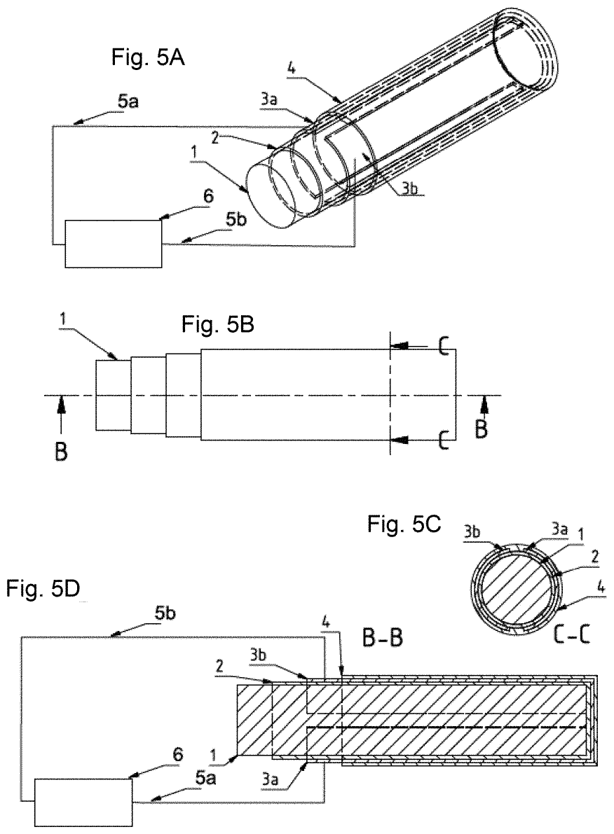

FIGS. 5A-5D show an alternative advantageous embodiment for a cylindrical object. FIG. 5A shows an overview of an exemplary cylindrical object, shown shortened here for reasons of the overview. The length is, however, generally freely selectable. Layers removed in steps at the end furthermore only serve the better understanding. The electrically conductive layer is divided into two part layers 3a and 3b here. They are electrically insulated from one another by the insulating layer 4. The connection to the control electronics takes place via the electrical contacts 5a and 5b respectively. The side view with the definition of the sections (C-C) and (B-B) is shown in FIG. 5B. FIG. 5C shows the sectional view (C-C) depicted in FIG. 5B. It can be seen here that the electrically conductive part layers 3a and 3b are insulated from one another by a gap. FIG. 5D shows the sectional view (B-B) depicted in FIG. 5B. It can be seen here that the electrically conductive part layers 3a and 3b are connected to one another at the end of the coated object 1 and the heating power circuit is thus closed. In comparison with an embodiment having only one electrically conductive layer, the electrical terminal 5 of the control electronics 6 can therefore advantageously only take place at one end of the object 1. In comparison with the embodiments shown in FIGS. 4A-4D, no additional layers are required for this purpose, however.

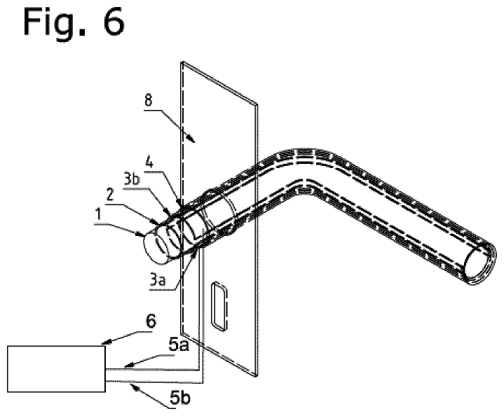

The advantageous embodiment shown in FIGS. 5A-5D is shown in the form of a door handle in FIG. 6. It can be heated to disinfect the surface by the provision at suitable time intervals in accordance with the invention. The disinfection takes place, for example, periodically or when the electronics determine a break in usage by a suitable sensor system, for instance by motion sensors or a proximity sensor system. An additional sensor system can generally also be integrated in the coating itself, e.g. in the form of an additional conductive layer whose capacitance, inductance, and resistance is detected. The door fitting 8 is also shown for illustration purposes in FIG. 6.

The advantageous embodiments shown in FIGS. 4A-4D and 5A-5D can furthermore be used for other cylindrical objects such as hand rails, handles, and grab handles.

1-15. (canceled)

16. A self-disinfecting object having a contact surface of the object provided for contact with a human body part, wherein the contact surface is at least regionally self-disinfecting in that at least one disinfection coating including at least one electrically conductive layer that is connected, for resistive heating of the at least one electrically conductive layer, via at least two electrical contacts to at least one control electronic system is arranged at least in regions of the contact surface.

17. The self-disinfecting object in accordance with claim 16,
wherein the control electronics have at least one temperature sensor and/or at least one temperature regulation.

18. The self-disinfecting object in accordance with claim 17, wherein the temperature regulation is adapted to a surface temperature of the object of at least 70° C.

19. The self-disinfecting object in accordance with claim 16,
wherein the at least one disinfection coating has at least one electrically insulating layer between the surface remote from the object and, starting from this, the next electrically conductive layer and/or has at least one thermally insulating layer between the surface facing the object and, starting from this, the next electrically conductive layer.

20. The self-disinfecting object in accordance with claim 16,
wherein the at least one electrically conductive layer comprises one of the following materials or combinations thereof:
metals;
carbon modifications;
electrically conductive ceramic materials; and
polymer materials comprising electrically conductive particles.

21. The self-disinfecting object in accordance with claim 16,
wherein the at least one electrically insulating layer comprises one of the following materials or combinations thereof:
thermally conductive materials;
polymer materials; and
polymer materials comprising particles.

22. The self-disinfecting object in accordance with claim 16,
wherein the at least one thermally insulating layer comprises one of the following materials or combinations thereof:
thermally insulating materials;
polymer materials comprising particles of thermally insulating materials;
materials comprising pores; and
materials comprising cavities.

23. The self-disinfecting object in accordance with claim 20,
wherein the polymer materials are selected from the group consisting of silicones and inorganic-organic hybrid polymers.

24. The self-disinfecting object in accordance with claim 20,
wherein the polymer materials are selected from the group consisting of organically modified polysiloxanes, fluorosilicone, polyurethane (PUR), ethylene propylene diene rubber, polychlorobutadiene (CR), acrylonitrilebutadiene (NBR), polyester resin, epoxy resin, acrylic resin, phenolic resin, amino resin, polyoxymethylenes (POMs), polypropylene (PP), acrylonitrile butadiene styrene copolymer (ABS), polycarbonate (PC), and polymethyl methacrylate (PMMA).

25. The self-disinfecting object in accordance with claim 16,
wherein the at least one electrically conductive layer has a uniform or locally varying layer thickness in the range from 0.1 to 1000 μm,
the at least one electrically insulating layer has a uniform or locally variable layer thickness in the range from 0.1 to 500 μm, and/or
the at least one thermally insulating layer has a uniform or locally variable layer thickness in the range from 0.05 to 10 mm.

26. The self-disinfecting object in accordance with claim 16,
wherein the coated contact surface has an irregular shape, with at least one of the electrically conductive layers on average becoming thicker in the direction of the narrower side and thinner in the direction of the wider side; and/or
at least one of the electrically conductive layers is divided into at least one strip that becomes wider in the direction of the narrower side and narrower in the direction of the wider side.

27. The self-disinfecting object in accordance with claim 16,
wherein the at least one electrically conductive layer has at least two segments electrically insulated from one another.

28. The self-disinfecting object in accordance with claim 16,
wherein at least one of the electrically conductive layers is formed as a resistive, capacitive, or inductive sensor element for measuring distance, temperature, pressure, elongation, force, shear, bend, or wetting of the contact surface or serves as a proximity sensor for living beings.

29. The self-disinfecting object in accordance with claim 16,
wherein the at least one disinfection coating has a transmission of at least 10% and/or at least one light element.

30. The self-disinfecting object in accordance with claim 16, wherein the disinfection coating is elastic.

31. The self-disinfecting object in accordance with claim 16, wherein the object is deformable.

32. The self-disinfecting object in accordance with claim 16,
wherein the object is selected from the group consisting of
manually operable devices;
mechanical holding and control devices;
switching or control elements;
medical devices; and
sports apparatus.
